(19) United States
(12) Patent Application Publication
(10) Pub. No.: US 2023/0410969 A1
(43) Pub. Date: Dec. 21, 2023

(54) PORTAL AND METHOD TO ENABLE A COMPLIANT COMMUNICATION PROTOCOL OVER A DISTRIBUTED NETWORK FOR THE E-TRANSFER OF PRESCRIPTIONS BETWEEN PHARMACIES

(71) Applicant: Four Leaf Rx, LLC D/B/A TransferMyRx, Plano, TX (US)

(72) Inventors: Nabil Adnan Hallak, Frisco, TX (US); Michael Lee Sands, Dallas, TX (US); Stephen Lanier Sands, Plano, TX (US); Mahdi Al Hallaq, Murphy, TX (US)

(21) Appl. No.: 18/209,911

(22) Filed: Jun. 14, 2023

Related U.S. Application Data
(60) Provisional application No. 63/352,493, filed on Jun. 15, 2022.

(51) Int. Cl. G16H 20/10; G16H 40/20

(52) U.S. Cl. CPC G16H 20/10; G16H 40/20; G16H 10/60

(57) ABSTRACT

A system for enabling an electronic transfer of a prescription order between pharmacies, comprises a first pharmacy and a second pharmacy different than the first pharmacy. A host server is in communication with the first and second pharmacies over a network and is configured to facilitate communication between the first and second pharmacies. The host server comprises a prescription transfer module configured to enable a prescription order to be exchanged between the first pharmacy and the second pharmacy.

### CROSS-REFERENCE TO RELATED APPLICATION(S)

The present application claims priority to U.S. Provisional Patent Application No. 63/352,493, entitled PORTAL AND METHOD TO ENABLE A COMPLIANT COMMUNICATION PROTOCOL OVER A DISTRIBUTED NETWORK FOR THE E-TRANSFER OF PRESCRIPTIONS BETWEEN PHARMACIES, filed on Jun. 15, 2022, the entirety of which is incorporated by reference herein.

### BACKGROUND

The present invention relates to compliant communication protocols over a distributed network and more particularly to compliant communication protocols over a distributed network between pharmacies.

Prescription control and accuracy are critical to the pharmacy industry. Advances in the industry have been relatively stagnant and there is a large unsolved need in the industry for technological developments that would enable pharmacists to do their job more efficiently and without compromising prescription accuracy and/or bypassing controls set in place to protect patients.

Presently, situations may arise where patients need to transfer their prescriptions to a different pharmacy, such as when, for example, traveling or simply because their insurance mandates the prescription be filled at a certain pharmacy. However, this may often become a tedious task. In order to have the prescription order transferred to a different pharmacy, patients must contact their pharmacy and request that the prescription be transferred. However, this process is antiquated, inefficient and problematic. In part, because it requires for the schedules of a pharmacist in the transferring pharmacy and a pharmacist in the receiving pharmacy to coincide between tasks and during a short window (e.g., a few hours)—so that both can direct time and attention to verifying a communication to convey the required information used to transfer the prescription. And regulations require that the transfer be performed by pharmacists. As such, there is a need to more easily, quickly, and efficiently streamline the process for pharmacies to transfer patient prescription orders between one another to provide more widespread access to patients in need of prescribed medication.

### SUMMARY OF THE INVENTION

Embodiments of the present invention address deficiencies of the art in respect to compliant communication protocol over a distributed network between pharmacies and provide a novel and non-obvious method, system or computer program product to enable a compliant communication protocol over a distributed network for the E-transfer of a prescription between unaffiliated pharmacies that do not share the same data.

According to one or more embodiments, a system for enabling an electronic transfer of a prescription order between pharmacies comprises a first pharmacy and a second pharmacy different than the first pharmacy. A host server is in communication with the first and second pharmacies over a network and is configured to facilitate communication between the first and second pharmacies. The host server comprises a prescription transfer module configured to enable a prescription order to be exchanged between the first pharmacy and the second pharmacy.

In one aspect, the first and second pharmacies exchange the prescription order via a web-based portal implemented by the system.

In another aspect, when exchanging the prescription order, at least one of the first and second pharmacy is configured to generate a prescription order transfer request.

In another aspect, the prescription order transfer request includes: identifying information for a patient; and a prescription order corresponding to the patient.

In another aspect, the prescription order transfer request is one of a Transfer In request and a Transfer Out Request.

In another aspect, the Transfer In request is one of: a request received by a receiving pharmacy to receive a prescription order from a transferring pharmacy; and a request sent by the receiving pharmacy to request that the prescription order be transferred by the transferring pharmacy.

In another aspect, the Transfer Out request is one of: a request sent by a transferring pharmacy requesting that a receiving pharmacy accepted the transfer of a prescription order from the transferring pharmacy and a request received by the transferring pharmacy requesting that the prescription order be sent to the receiving pharmacy.

In another aspect, one of the first pharmacy and the second pharmacy is a transferring pharmacy and the other is a receiving pharmacy.

In another aspect, the prescription order transfer request is generated by at least one of the transferring pharmacy and receiving pharmacy via a computing device configured to receive input from a user. The computing device is in communication with the web-based portion.

In another aspect, upon receipt of the prescription order transfer request, the prescription transfer module is configured to receive input from the computing device of the user. The input indicates whether at least one of the transferring and receiving pharmacies accepts or declines the request.

According to one or more other embodiments, a prescription order transfer system maintains a web-based portal. The prescription order transfer system comprises a network, a plurality of computing devices in communication with each other over the network; and a prescription transfer module configured to enable a prescription order transfer request to be exchanged between the plurality of computing devices.

In one aspect, the plurality of computing devices comprises a computing device of a transferring pharmacy and a computing device of a receiving pharmacy.

In another aspect, upon receipt of a prescription order transfer request, one of the transferring pharmacy and the receiving pharmacy may accept or decline the request.

In another aspect, the prescription transfer module is configured to generate and send a notification to the transferring and receiving pharmacies indicating whether the prescription order transfer request has been accepted or declined.

In another aspect, upon acceptance of the prescription order transfer request, the prescription order transfer module generates a transfer summary that includes a set of information associated with the prescription order. The set of information is presented to each of the transferring and receiving pharmacies via a display of their respective computing devices.

In another aspect, the prescription order transfer request includes identifying information for a patient and a prescription order corresponding to the patient.

In another aspect, the Transfer In request is one of a request received by a receiving pharmacy to receive a prescription order from a transferring pharmacy and a request sent by the receiving pharmacy to request that the prescription order be transferred by the transferring pharmacy.

In another aspect, the Transfer Out request is one of a request sent by a transferring pharmacy requesting that a receiving pharmacy accepted the transfer of a prescription order from the transferring pharmacy and a request received by the transferring pharmacy requesting that the prescription order be sent to the receiving pharmacy.

In another aspect, the server is configured to log a record of each Transfer In and Transfer Out request received and sent by each pharmacy.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 shows an exemplary prescription order transfer system, in accordance with the principles of the present application;

FIG. 2 shows an exemplary computing device of the system of FIG. 1, in accordance with the principles of the present application;

FIG. 3 shows an exemplary server of the system of FIG. 1, in accordance with the principles of the present application;

FIG. 4 shows an exemplary “Transfer In” request form generated by the system of FIG. 1, in accordance with the principles of the present application;

FIG. 5 shows a full list of “Transfer In” requests sent or received by a user pharmacy, in accordance with the principles of the present application;

FIG. 6 shows an exemplary “Transfer Out” request form generated by the system of FIG. 1, in accordance with the principles of the present application;

FIG. 7 shows a full list of “Transfer Out” requests sent or received by a user pharmacy, in accordance with the principles of the present application;

FIG. 8 shows an exemplary Transfer Out request acceptance summary, in accordance with the principles of the present application; and

FIG. 9 shows an exemplary controlled substance checkpoint alert generated by the system of FIG. 1, in accordance with the principles of the present application.

### DETAILED DESCRIPTION

Embodiments of the invention provide systems and methods for an electronic platform that enables a compliant communication protocol over a distributed network for the electronic transfer (“E-transfer”) of a prescription between pharmacists at unaffiliated pharmacies that do not share the same data allowing the pharmacists to securely, accurately and efficiently communicate electronically. According to an embodiment of the invention, the invention is directed to unaffiliated pharmacies that do not share all of the same data because pharmacies that share certain data are excluded from the prescription transfer regulations and may transfer prescriptions electronically without any restrictions. In order to enable the transfer of prescription between unaffiliated pharmacies that do not share the same data, the system may communicate with the databases of different unaffiliated pharmacies that do not share data with each other or may allow pharmacists of unaffiliated pharmacies that have access to their respective pharmacy database to securely communicate with each other. Each of the databases store the respective inventory of available prescriptions and patient information for the respective pharmacy.

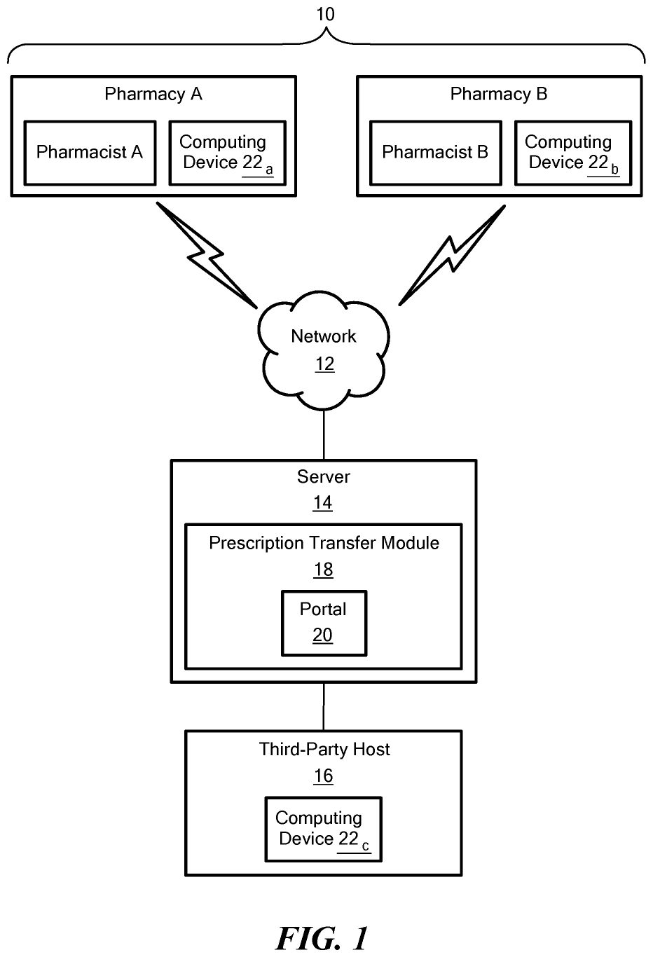

Referring now to the drawings in which like reference designators refer to like elements, there is shown in FIG. 1, a computer-implemented prescription order transfer system 10 that enables a compliant communication protocol between one or more pharmacies, in accordance with principles of the present application. As shown in FIG. 1, the system 10 comprises one or more pharmacies, such as Pharmacy A and Pharmacy B who are members of the system 10. The pharmacies may communicate over network 12 and are each in communication with a host server 14 managed by a third-party host 16. In one or more embodiments, the host sever 14 serves as an intermediary and facilitates communication between each pharmacy through the use of a prescription order transfer module 18 that provides access to a web-based member portal 20. Pharmacy A may include a user or Pharmacist A and a computing device 22ₐ configured to receive input from the Pharmacist A. Similarly, the Pharmacy B may include a second user or Pharmacist B and a computing device 22_{b} configured to receive input from Pharmacist B. The host server 14 may also be accessed by computing device 22_{c} of the host 16.

Continuing to refer to FIG. 1, as described herein the prescription order transfer module 18 is configured to allow each pharmacy to send and/or receive prescription orders and prescription order transfer requests to and from one or more other pharmacies via the portal 20. Thus, it is to be understood that all system functions, operations, and processes necessary to facilitate the prescription order transfer requests described herein, approvals of the prescription order transfer requests, and all other system functions of the portal presented to user pharmacist via the portal 20, are possible through the use of the prescription transfer module 18.

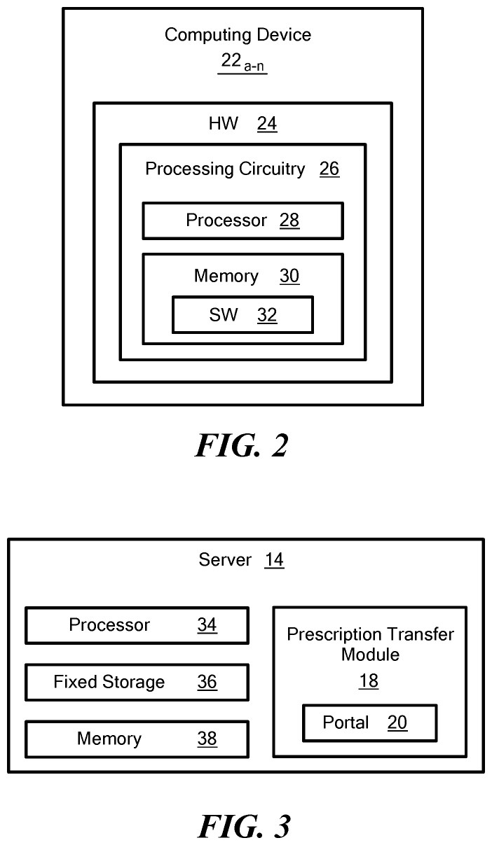

As shown in FIG. 2, computing device 22ₐ₋ₙ further includes hardware and software configured to perform and execute the operations, functions, and processing necessary to implement the system described herein. In some embodiments, the hardware (HW) 24 may include processing circuitry 26 that comprises a processor 28 and a memory 30 in communication with the processor 28. In particular, in addition to or instead of a processor, such as a central processing unit and memory, the processing circuitry 26 may include integrated circuitry for processing and/or control, e.g., one or more processors and/or processor cores and/or FPGAs (Field Programmable Gate Array) and/or ASIC's (Application Specific Integrated Circuitry) adapted or configured to execute instructions. The processor 28 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Further, memory 30 may be configured as a storage device.

The processing circuitry 26 may be configured to control any of the methods and/or processes described herein and/or to cause such methods and/or processes to be performed by the computing devices 22_{a_n} described herein. Processor corresponds to one or more processors for performing the computing device functions described herein. In some embodiments, the software 32 may include instructions that, when executed by the processor 28 and/or processing circuitry 26, causes the processor 28 and/or processing circuitry 26 to perform the processes described herein with respect to the computing devices 22ₐ₋ₙ.

The software 32 may be stored internally in, for example, memory 30, or stored in external memory (e.g., database, storage array, network storage device, etc.) and accessible via an external connection. The software 32 may be executable by the processing circuitry 26.

Now referring to FIG. 3, in one or more embodiments the server 14 may include a processor 34, a fixed storage 36, memory 38 in communication with the processor 34 and/or fixed storage 36, and the prescription transfer module 18 that executes the portal 20. In particular, in addition to or instead of a processor, such as a central processing unit and memory, the server 14 may include integrated circuitry for processing and/or control, e.g., one or more processors and/or processor cores and/or FPGAs (Field Programmable Gate Array) and/or ASIC's (Application Specific Integrated Circuitry) adapted or configured to execute instructions. The processor 34 may be configured to access (e.g., write to and/or read from) the memory 38, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Further, memory 38 may be configured as a storage device in addition to or in place of fixed storage 36.

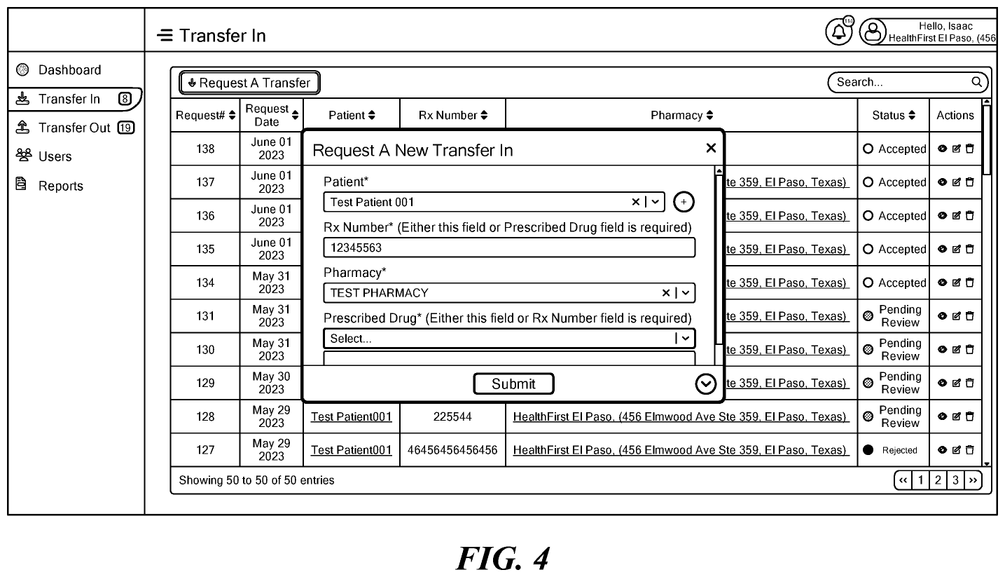

The present system 10 provides a way for pharmacies to quickly and easily communicate with one another when requesting to receive a customer or patient's prescription from another pharmacy, or requesting to send the prescription order to another pharmacy. As an example, in certain situations, upon receiving a prescription order from a patient's prescriber, the patient's existing pharmacy (ex: Pharmacy A) may realize that it cannot fulfill the prescription (e.g., drug out of stock) or the patient may desire the prescription order be transferred to a different pharmacy (ex: Pharmacy B). Pharmacy A may access the portal 20 via their computing device 22ₐ (which is in communication with server 14) to transfer the prescription order (i.e., “Transfer Out” discussed in more detail below) to Pharmacy B that may be able to fulfill the order. Once Pharmacist A identifies a Pharmacy B suitable for receiving the prescription order, it may submit a prescription transfer request through the portal 20 to Pharmacy B, which also has access to the portal 20 via its computing device 22b. Pharmacy B will be notified of the transfer request by a notification alert that will be generated on Pharmacy B's portal account dashboard. It is to be understood that the prescription transfer request may include data and information associated with the patient, details for a particular prescription drug for the patient, and a request for Pharmacy B to accept transfer of the prescription order. Upon being notified that it has received a prescription transfer request, Pharmacist B may navigate to a request page (see FIG. 4) where it may review the details of the prescription transfer request. Upon review, Pharmacist B may then determine if it would like to accept transfer of the prescription order or not. The portal 20 receives user input from Pharmacist B indicating whether the request has been accepted or declined. Upon acceptance or denial, the server 14 generates a notification to alert Pharmacy A of Pharmacy B's decision. If Pharmacy B does accept transfer of the prescription order, the prescription order is immediately available for Pharmacy B to review and process.

As described herein, it is to be understood that the prescription transfer request is accessible and displayed to each user or pharmacist via their respective computing device 22. The prescription transfer request, any subsequent prescription transfers, and any communication between the pharmacies may be conducted over network 12 through the server 14.

Now referring to FIG. 4, a “Transfer In” request form in shown. The system 10 is configured to allow a member pharmacy (i.e., a pharmacy registered to use the portal 20) to “transfer in” and “transfer out” a patient's prescription. For example, the system 10 may generate a fillable web-based form for a user pharmacist to fill out when requesting a “transfer in” of a prescription from another pharmacy. A “Transfer In” is a request by a pharmacy for a prescription order to be transferred to it from another pharmacy. This may apply in situations where the patient is not near the pharmacy and is in need of their prescription. The patient may ask for a nearby pharmacy to request that the prescription order be sent to it so that the patient can have access to their prescription. However, this may also be applicable in a variety of situations such as, for example, a patient simply wants to use a different pharmacy. However, as described herein, a “Transfer In” may also be when a first pharmacy receives a request from a second pharmacy for the second pharmacy to transfer a prescription to the first pharmacy. Similarly, a “Transfer Out” request may refer to situations where a pharmacy wishes to send the prescription order out to another pharmacy or has received a request from another pharmacy to transfer a prescription to it.

As shown in FIG. 4, the generated “Transfer In” form may request certain information to be provided by the requesting pharmacist. As a non-limiting example, the requested information may include patient name, pharmacy name, prescription (Rx) number, the prescribed drug and dosage, etc. Once the form has been completed, the system 10 may accept input from the user to send the request to the patient's current pharmacy who is also registered to use the portal 20. In this example, the pharmacy requesting the transfer in is the “receiving pharmacy” and the pharmacy who would be approving the request is the “transferring pharmacy.” Upon receipt of the transfer in request, the transferring pharmacist may review the request and enter input to accept or decline the request. If accepted, the prescription order will be automatically transferred by the system 10 to receiving pharmacy.

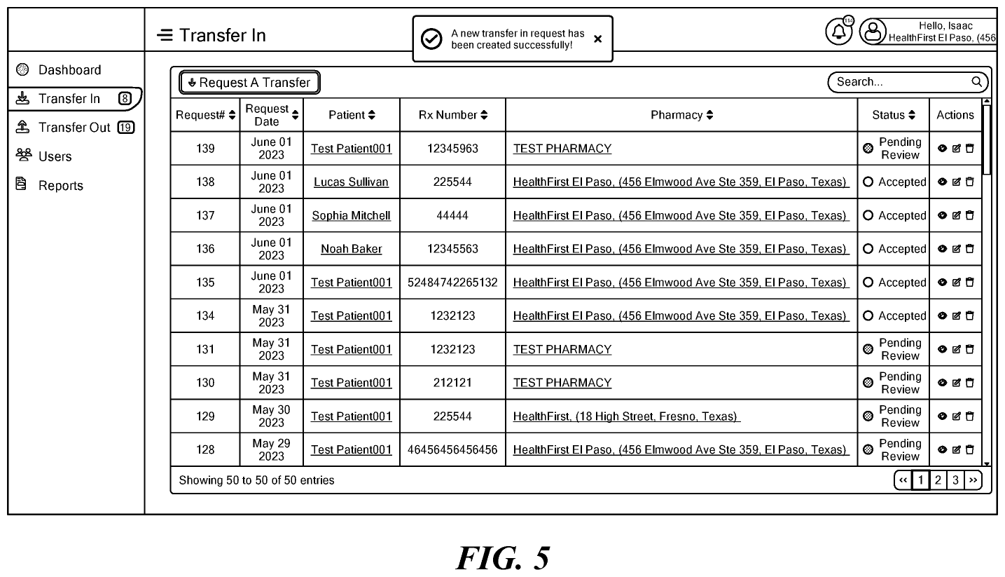

As shown in FIG. 5, the system 10 is also capable of populating a full list of transfer in requests—whether initiated by receiving pharmacy or not. This list shows all of the transfer in request created by the current user pharmacy, as well as the requests received from outside pharmacies. The system 10 shows certain information such as the request number, request date, patient name, Rx number, pharmacy name, status, and action items.

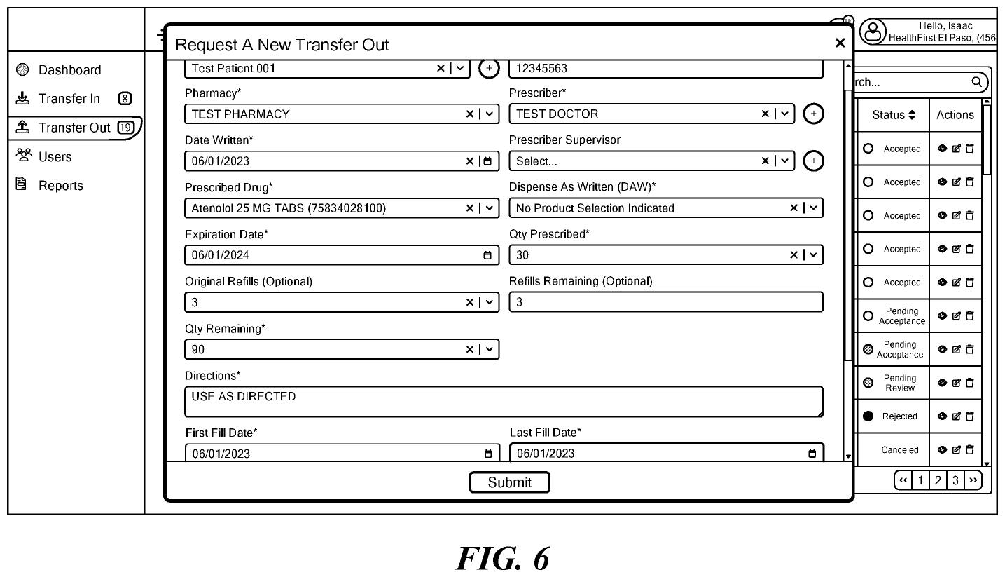

As shown in FIG. 6, the system 10 is also capable of generating “Transfer Out” forms that require transferring pharmacists to enter various information such as, for example, patient name, Rx number, pharmacy name, prescriber name, date written, prescriber supervisor, prescribed drug, dispense as written, expiration date, quantity prescribed, refill amount, refills remaining, quantity remaining, directions, first fill date, and last fill date, etc. Once completed, the transferring pharmacist may enter user input via their computing device 22 to initiate the transfer of the prescription order request to the receiving pharmacy who may then accept or decline the request. If accepted, the receiving pharmacy will be provided with the prescription order and the transferring pharmacy will be alerted via a portal notification of the acceptance. If declined, the transferring pharmacy will be alerted via a notification that the request has been denied. The transferring pharmacy may then generate a new Transfer Out request to send to a different receiving pharmacy.

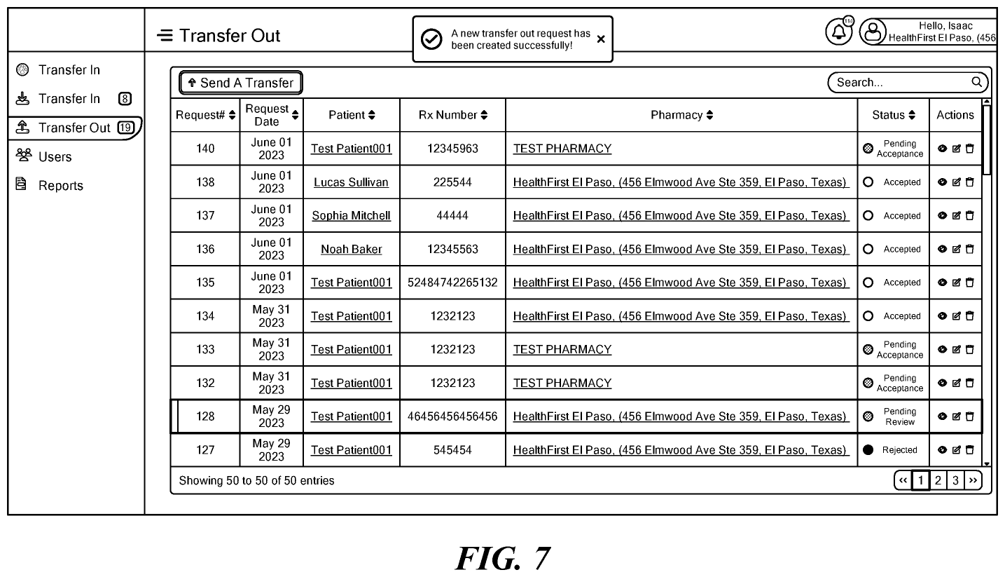

As shown in FIG. 7, the system 10 is also capable of populating a full list of Transfer Out requests—whether initiated by transferring pharmacy or not. This list shows all of the Transfer Out requests created by the current user pharmacy, as well as the requests received from outside pharmacies. The system 10 shows certain information such as the request number, request date, patient name, Rx number, pharmacy name, status, and action items.

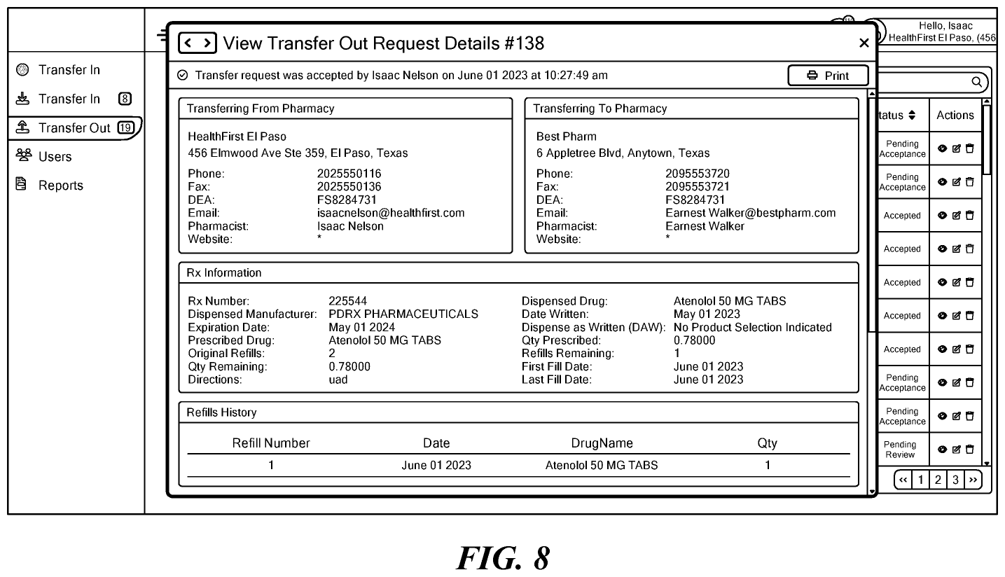

Now referring to FIG. 8, an exemplary Transfer Out request acceptance summary is shown. Upon acceptance of a Transfer Out request, the system 10 is capable of generating a summary that shows the full details associated with the prescription transfer to the other pharmacy. This summary may be downloaded or printed by a pharmacist onto their computing device 22ₐ₋ₙ.

According to one or more embodiments, the pharmacist of each respective pharmacy may manually check their inventory to determine whether a prescribed drug is in stock. This may affect whether the pharmacist chooses to accept or decline a Transfer In request, or initiate a Transfer Out request. However, it is to be understood that in some other embodiments, the system 10 may be configured to access a pharmacies inventory database to automatically determine whether a particular drug is in stock. For example, if a pharmacy wishes to transfer the prescription order out because it does not have it in stock, the system 10 may automatically access and check the inventories of other pharmacies to determine which pharmacies have the drug in stock. Once the system 10 has identified the pharmacies having the drug, the system 10 may alert the transferring pharmacy as to which pharmacies it may transfer the prescription order to.

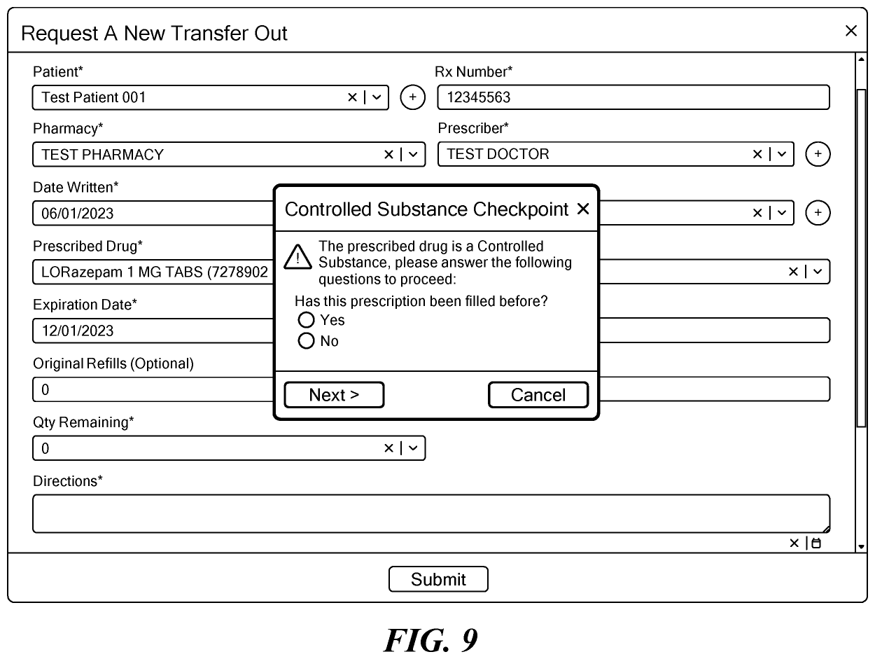

Now referring to FIG. 9, in some embodiments the system 10 is further capable of generating a “Controlled Substance Checkpoint” alert which is presented to a user pharmacist when preparing a Transfer Out request so that the transfer may comply with local or federal regulations. The checkpoint requires the pharmacist to enter additional information when requesting to transfer out controlled substances. Before accepting the Transfer Out Request, the receiving pharmacist may manually check and confirm inventory, and check the Prescription Monitoring Program (PMP) system for control substances abuse, etc. After confirmation by the transferring pharmacists and/or receiving pharmacist, the prescription is then transferred to the receiving pharmacy.

The system 10 may also send a notification at any point during the transfer process to the transferring pharmacy, receiving pharmacy and/or patient regarding the status of the prescription transfer. As an example, the notification may notify the receiving pharmacy that the prescription transfer is waiting for confirmation or to set up secure communication to transfer the prescription. The notification may include a key for the pharmacist of either pharmacy, which may be made up letters and/or numbers or any combination of characters. The key may be used by the notified pharmacist to ensure that a record is maintained in the database regarding the transfer and parts of the transfer in which the notified pharmacist was involved. As described herein, it is to be understood that any and all alerts, notifications, warnings, or messages generated by the system 10 may be displayed to the user pharmacist via a display of their respective computing device 22. The user pharmacist may interact with any necessary system features through the use of a user interface configured to accept input from the user on the computing device 22.

In some embodiments, the system 10 may also include pharmacy location-based timing thresholds and escalation procedures in order to coordinate the scheduling of pharmacists to transfer prescriptions. The calendars for pharmacists may be stored in order to schedule transfer requests to avoid wasting a pharmacist's time. There may also be escalation procedures, such as urgent notifications, to expedite transfer of the prescription for a patient for any reason. Further, the timing thresholds and escalation procedures may be based on Federal or State Law requirements that require a prescription transfer to occur within a certain time frame. As an example, in order to transfer controlled substances, there is often a time limit—normally, 24 hours but any amount of time is within the scope of this invention. Therefore, the system 10 may provide notifications to ensure the time limit for the transfer of controlled substances is met. The system 10 may also record data as to when certain actions were performed, such as when the request to transfer was entered, when the receiving pharmacy was notified, when the transfer was completed, etc. in order to provide a time trail of what happened, which may be utilized for audit and/or law enforcement purposes and enforcing law.

Further, in some embodiments, the pharmacists and pharmacies may be verified before establishing secure communication and before checking the inventory of the receiving pharmacy in any up-to-date pharmacies database to ensure the pharmacists/pharmacies are verified for transfer. As well, all communications between the databases and pharmacies are secure and encrypted so that the entire transfer is HIPAA compliant. The system 10 may also communicate with controlled substances databases, such as the Drug Enforcement Agency (DEA) or PMP controlled substance databases to display any information regarding the prescription and patient or to update the database after transfer of the prescription. For controlled substance transfers, there may be a questionnaire prompt to either of the pharmacists to verify that the prescription qualifies for a legal transfer as State and Federal law restrict the control substances prescriptions transfer, which will help in reducing controlled substances fraud.

The system 10 may be capable of capturing real-time, time-stamped documentation of communications between pharmacists approving or denying the transfer or any of the steps of the transfer, including any notifications, which will help pharmacists adhere to Federal and State laws that limit the amount of time that is allowed in order for a prescription transfer to be executed. Further, the system 10 may allow a consumer/patient to send their prescription transfer requests securely and directly to the portal in a HIPPA compliant fashion in order to automatically initiate the prescription transfer.

Thus, the system 10 provides an improvement to the transfer of prescriptions through verification of pharmacists, pharmacy location-based timing thresholds and escalation procedures, HIPPA compliant, electronic transfer of information, automated inventory verification, improved pharmacists' and patients' experiences and prescription tracking, and/or optimized controlled substance database review. The system 10 also provides an improvement in the accuracy aspect of transferring of prescription as transfer will not need to be done verbally, which creates a lot of “he said she said” situations as there is no written confirmation in addition to the verbal confirmation, and instead creates a paper trail of requests for transfer, confirmations, notifications, etc. Further, the system 10 will improve patients' lives by getting the patient's medications faster and with less potential for errors.

According to one or more embodiments, a method, system or computer program product to enable a compliant communication protocol over a distributed network for the E-transfer of a prescription between pharmacies includes communicating with a first database, comprising an inventory of a transferring pharmacy, wherein the inventory of the transferring pharmacy comprises a list of prescriptions available at the transferring pharmacy and communicating with a second database comprising an inventory of a receiving pharmacy that is different than the transferring pharmacy, wherein the inventory of the receiving pharmacy comprises a list of prescriptions available at the receiving pharmacy. The method further includes receiving a prescription transfer request from the transferring pharmacy to the receiving pharmacy, wherein the prescription transfer request comprises a prescription and verifying the prescription is available in the inventory of the receiving pharmacy. The method further includes responsive to verifying that the prescription is available in the inventory of the receiving pharmacy, establishing a secure communication between a pharmacist of the transferring pharmacy and a pharmacist of the receiving pharmacy, prompting the pharmacist of the transferring pharmacy to confirm a transfer of the prescription, and responsive to the pharmacist of the transferring pharmacy confirming transfer of the prescription, transferring the prescription to the receiving pharmacy.

According to one or more embodiments, the pharmacist of the transferring pharmacy and the pharmacist of the receiving pharmacy are verified in a NPI or NCPDP database. In another aspect of the embodiment, the transferring of the prescription from the transferring pharmacy to the receiving pharmacy is HIPAA compliant. In yet another aspect of the embodiment, the method further includes communicating with a controlled substance database, displaying information regarding the prescription from the controlled substance database, and automatically updating the controlled substance database after transferring the prescription to the receiving pharmacy. In even yet another aspect of the embodiment, the method further includes storing a calendar for the pharmacist at the transferring pharmacy, storing a calendar for the pharmacist at the receiving pharmacy, and further responsive to verifying that the prescription is available in the inventory of the receiving pharmacy: scheduling a time to establish the secure communication between the pharmacist of the transferring pharmacy and the pharmacist of the receiving pharmacy in the calendar of the pharmacist at the transferring pharmacy and the calendar for the pharmacist at the receiving pharmacy. In another aspect of the embodiment, the scheduling a time to establish the secure communication between the pharmacist of the transferring pharmacy and the pharmacist of the receiving pharmacy is escalated to expedite transfer of the prescription to the receiving pharmacy.

According to one or more embodiments, a data processing system configured to enable a compliant communication protocol over a distributed network for the E-transfer of a prescription between pharmacies includes a host computing system including one or more computers each with memory and at least one processor. The host computing system is in communication with a first database storing an inventory of a transferring pharmacy, wherein the inventory of the transferring pharmacy comprises a list of prescriptions available at the transferring pharmacy and the host computing system is in communication with a second database storing an inventory of a receiving pharmacy that is different than the transferring pharmacy, wherein the inventory of the receiving pharmacy comprises a list of prescriptions available at the receiving pharmacy. The system further includes an application executing in memory of the host computing system and a compliant prescription transfer module coupled to the application. The module includes program code enabled to receive or initiate a prescription transfer request from the transferring pharmacy transferring pharmacy or receiving pharmacy, establishing a secure communication between a pharmacist of the transferring pharmacy and a pharmacist of the receiving pharmacy, prompting the pharmacist of the transferring pharmacy to confirm a transfer of the prescription or prompting the pharmacist of the receiving pharmacy to accept transfer of the prescription; and generating a notification to each pharmacist regarding whether the transfer request has been accepted or declined.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A system for enabling an electronic transfer of a prescription order between pharmacies, the system comprising:
a first pharmacy and a second pharmacy different than the first pharmacy;
a host server in communication with the first and second pharmacies over a network and configured to facilitate communication between the first and second pharmacies; and
wherein the host server comprises a prescription transfer module configured to enable a prescription order to be exchanged between the first pharmacy and the second pharmacy.

2. The system of claim 1, where the first and second pharmacies exchange the prescription order via a web-based portal implemented by the system.

3. The system of claim 1, wherein when exchanging the prescription order, at least one of the first and second pharmacy is configured to generate a prescription order transfer request.

4. The system of claim 3, wherein the prescription order transfer request includes:
identifying information for a patient; and
a prescription order corresponding to the patient.

5. The system of claim 4, wherein the prescription order transfer request is one of a Transfer In request and a Transfer Out Request.

6. The system of claim 5, wherein the Transfer In request is one of:
a request received by a receiving pharmacy to receive a prescription order from a transferring pharmacy; and
a request sent by the receiving pharmacy to request that the prescription order be transferred by the transferring pharmacy.

7. The system of claim 5, wherein the Transfer Out request is one of:
a request sent by a transferring pharmacy requesting that a receiving pharmacy accepted the transfer of a prescription order from the transferring pharmacy; and
a request received by the transferring pharmacy requesting that the prescription order be sent to the receiving pharmacy.

8. The system of claim 5, wherein one of the first pharmacy and the second pharmacy is a transferring pharmacy and the other is a receiving pharmacy.

9. The system of claim 8, wherein the prescription order transfer request is generated by at least one of the transferring pharmacy and receiving pharmacy via a computing device configured to receive input from a user, the computing device is in communication with the web-based portion.

10. The system of claim 8, wherein upon receipt of the prescription order transfer request, the prescription transfer module is configured to receive input from the computing device of the user, the input indicating whether at least one of the transferring and receiving pharmacies accepts or declines the request.

11. A prescription order transfer system, the prescription order transfer system maintaining a web-based portal, the prescription order transfer system comprising:
a network;
a plurality of computing devices in communication with each other over the network; and
a prescription transfer module configured to enable a prescription order transfer request to be exchanged between the plurality of computing devices.

12. The system of claim 11, wherein the plurality of computing devices comprises a computing device of a transferring pharmacy and a computing device of a receiving pharmacy.

13. The system of claim 12, wherein upon receipt of a prescription order transfer request, one of the transferring pharmacy and the receiving pharmacy may accept or decline the request.

14. The system of claim 13, wherein the prescription transfer module is configured to generate and send a notification to the transferring and receiving pharmacies indicating whether the prescription order transfer request has been accepted or declined.

15. The system of claim 14, wherein upon acceptance of the prescription order transfer request, the prescription order transfer module generates a transfer summary that includes a set of information associated with the prescription order, the set of information is presented to each of the transferring and receiving pharmacies via a display of their respective computing devices.

16. The system of claim 11, wherein the prescription order transfer request includes:
identifying information for a patient; and
a prescription order corresponding to the patient.

17. The system of claim 16, wherein the prescription order transfer request is one of a Transfer In request and a Transfer Out Request.

18. The system of claim 17, wherein the Transfer In request is one of:
a request received by a receiving pharmacy to receive a prescription order from a transferring pharmacy; and
a request sent by the receiving pharmacy to request that the prescription order be transferred by the transferring pharmacy.

19. The system of claim 17, wherein the Transfer Out request is one of:
a request sent by a transferring pharmacy requesting that a receiving pharmacy accepted the transfer of a prescription order from the transferring pharmacy; and
a request received by the transferring pharmacy requesting that the prescription order be sent to the receiving pharmacy.

20. The system of claim 17, wherein the server is configured to log a record of each Transfer In and Transfer Out request received and sent by each pharmacy.
